(19) United States
(12) Patent Application Publication
(10) Pub. No.: US 2016/0038125 A1
(43) Pub. Date: Feb. 11, 2016

(54) GUIDED SEMIAUTOMATIC ALIGNMENT OF ULTRASOUND VOLUMES

(71) Applicant: General Electric Company, Schenectady, NY (US)

(72) Inventors: Andreas Haas, Schorfling (AT); Christian Zillner, Geiersberg (AT); Seyed Ebrahim Dehghani Bonaki, Salzburg (AT); Christian Stoeckl, Kufstein (AT)

(21) Appl. No.: 14/453,558

(22) Filed: Aug. 6, 2014

(51) Int. Cl. A61B 8/08; G06T 7/00; G06K 9/46; A61B 8/00; A61B 8/14

(52) U.S. Cl. CPC A61B 8/5253; A61B 8/463; A61B 8/0866; A61B 8/0883; A61B 8/145; A61B 8/466; A61B 8/467; A61B 8/4444; G06K 9/46; G06T 7/0028; G06T 2207/10136; G06T 2207/30048

(57) ABSTRACT

Embodiments for aligning a volume to a standard alignment are provided. On example method includes accessing three images, each image representing a respective plane of three intersecting planes of a volume, identifying a feature of interest in each of the three images, outputting one or more guidance indicators that indicate how the three images are to be aligned with respect to the feature of interest to correspond with the standard orientation, and adjusting the three images according to the one or more guidance indicators.

### FIELD

Embodiments of the subject matter disclosed herein relate to an ultrasound system, for example.

### BACKGROUND

During an ultrasound imaging session, 3D/4D volumes may be acquired in order to enable viewing of desired anatomical features that may be obstructed or otherwise difficult to view in traditional 2D imaging. After generating a 3D volume of a desired anatomy, the volume may be aligned to a standard alignment to facilitate location of the desired anatomical features. However, such alignment procedures may be time consuming and may require a high level of experience with ultrasound imaging techniques and the anatomy being imaged.

### BRIEF DESCRIPTION

In one embodiment, a method of aligning a volume to a standard orientation includes accessing three images, each image representing a respective plane of three intersecting planes of the volume, identifying a feature of interest in each of the three images, outputting one or more guidance indicators that indicate how the three images are to be aligned with respect to the feature of interest to correspond with the standard orientation, and adjusting the three images according to the one or more guidance indicators.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasonic imaging system.

FIG. 2 shows a flow chart illustrating an example method for aligning a volume with respect to feature of interest of the volume.

FIG. 3 shows a flow chart illustrating an example method for aligning a three-dimensional volume of a heart with respect to an interventricular septum of the heart.

FIGS. 4-8 are example graphical user interfaces output during execution of the method of FIG. 3.

### DETAILED DESCRIPTION

The following description relates to various embodiments of aligning a volume to a standard orientation. The alignment may include accessing, for a given volume, images representing three intersecting planes of the volume that include a feature of interest of the volume. One or more guidance indicators may be output that textually or graphically illustrate to an operator, for example, how the images are to be adjusted so that the feature of interest is aligned with respect to the standard orientation. The images may be adjusted with respect to the feature of interest, thus aligning the volume to the standard orientation. The volume may include a suitable volume, such as an anatomical structure (e.g., an organ such as the heart, brain, liver, etc.) reconstructed from a plurality of ultrasound images, for example. The feature of interest may be a sub-anatomical structure within the volume. In one example, the volume may be a heart and the feature of interest may be an interventricular septum. By aligning the volume in three planes to a feature of interest, a fast, easily reproducible alignment may be achieved. Standard alignment of the imaged volume (e.g., a heart) may facilitate further diagnostics, including the identification of diagnostically-relevant image planes of the heart, calculation of blood flow/pumping rates, or other suitable diagnostics.

While alignment of an imaged heart of an adult patient may be relatively easy, due to the patient assuming a standard orientation during imaging, alignment of a fetal heart may be particularly challenging due to the changing and unknown orientation of the fetus during imaging. Previous fetal heart alignment strategies relied on identification of an anatomical feature outside the heart that is easy to identify and orient, such as the fetal spine. However, the spine is not necessarily in the same position relative to the heart in all fetuses. To compensate for the differing anatomies among fetuses, tomographic ultrasound imaging (TUI) may be used during heart alignment with respect to the spine. TUI provides nine views of a slice of a target region of the fetus, thus requiring numerous steps of rotation and translation of the volume, necessitating that the operator have a high level of understanding of the alignment routine and anatomy of the fetus.

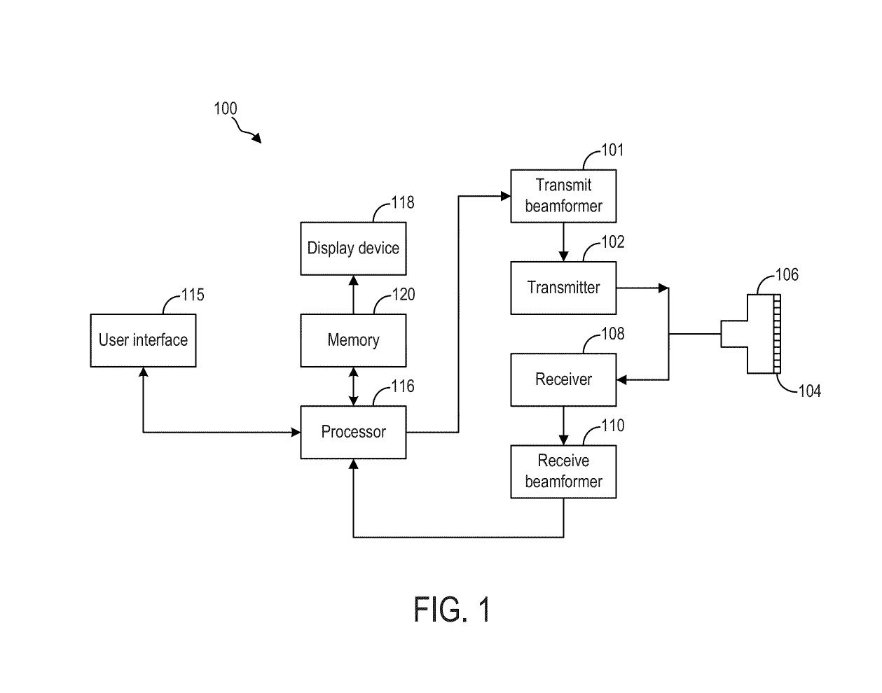

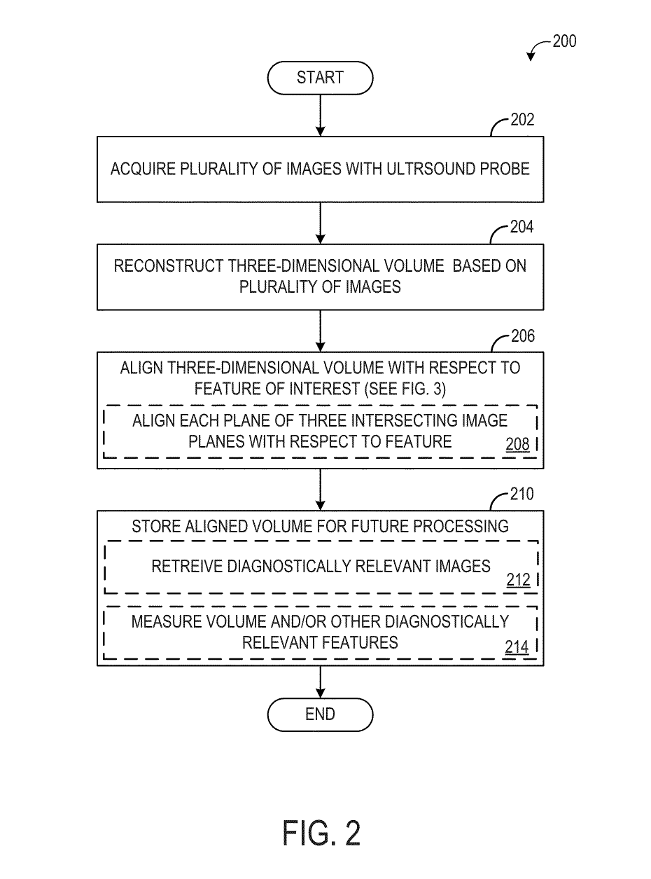

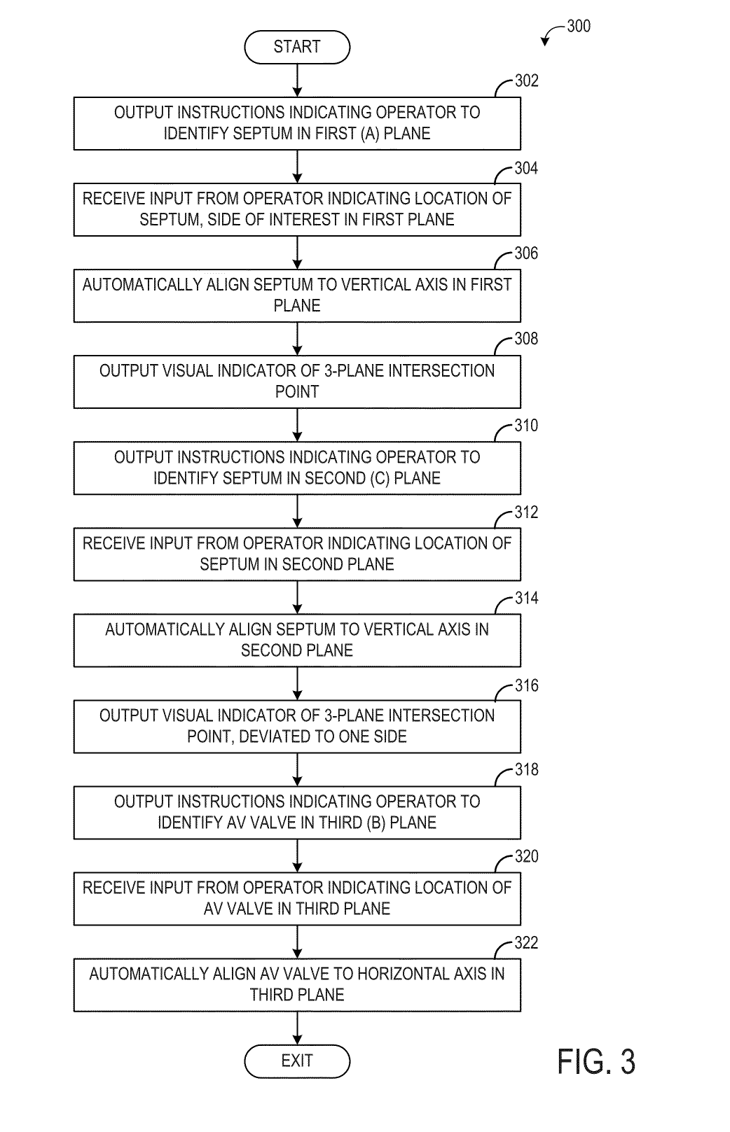

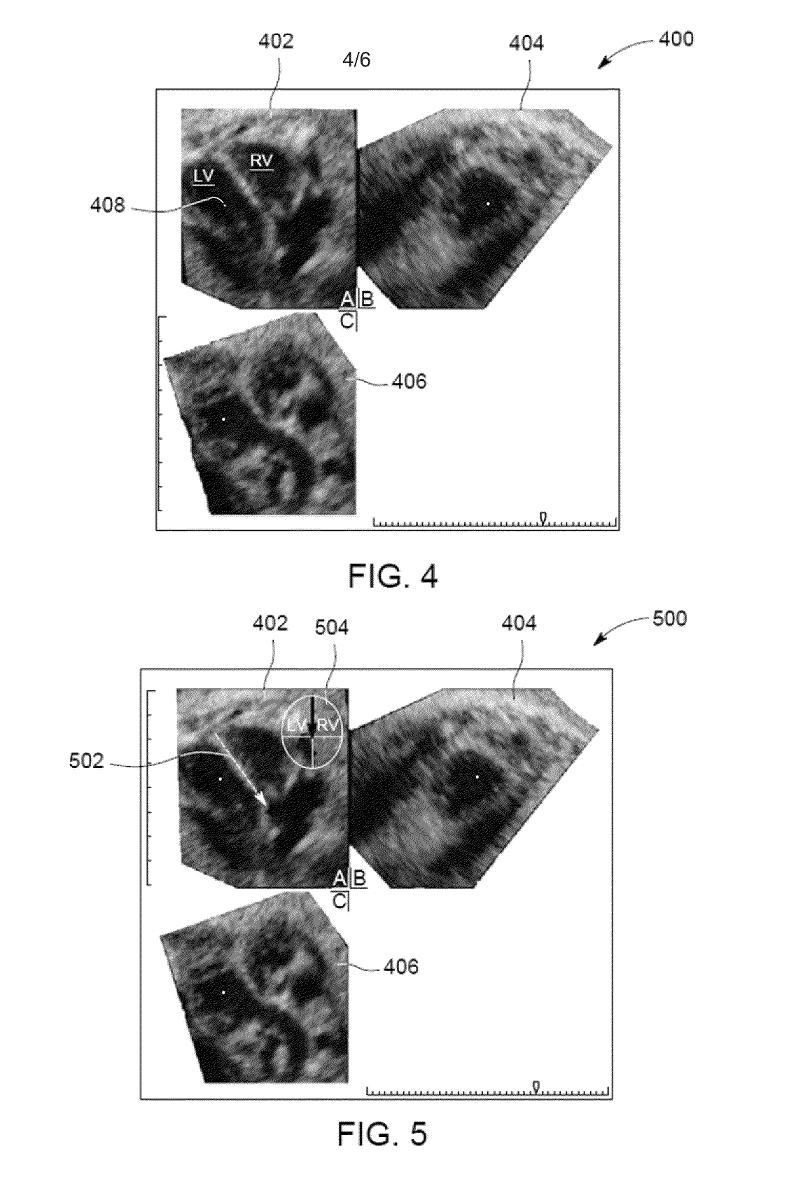

According to embodiments disclosed herein, a volume reconstructed from a plurality of ultrasound images may be aligned to a standard alignment using an anatomical feature within the volume itself. By aligning the volume with respect to a feature of the volume, differences in the relative positioning between the volume and the orientation landmark (e.g., the feature of the volume) may be minimized, and an accurate alignment can be provided using only three planes of the volume. FIG. 1 shows an ultrasound system that may be used to acquire the images for reconstructing a three-dimensional volume of a target region. The ultrasound system of FIG. 1 also includes a computing system including instructions to carry out one or more alignment routines to provide a guided, semi-automatic alignment of the volume. FIGS. 2-3 are flow charts illustrating methods that may be carried out by the computing system of the ultrasound system of FIG. 1. FIGS. 4-8 illustrate example graphical user interfaces output by the ultrasound system of FIG. 1, including intersecting planes of a volume of a heart during the alignment process of the method of FIG. 3.

FIG. 1 is a schematic diagram of an ultrasound imaging system 100 in accordance with an embodiment of the invention. The ultrasound imaging system 100 includes a transmit beamformer 101 and a transmitter 102 that drive transducer elements 104 within a probe 106 to emit pulsed ultrasonic signals into a body (not shown). A variety of geometries of probes and transducer elements may be used. The pulsed ultrasonic signals are back-scattered from structures in the body, such as blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system.

A user interface 115 may be used to control operation of the ultrasound imaging system 100, including controlling the input of patient data, changing a scanning or display parameter, and the like. The user interface 115 may include a graphical user interface configured for display on a display device 118. The graphical user interface may include information to be output to a user (such as ultrasound images, patient data, etc.) and may also include menus or other elements through which a user may enter input to the computing system. In examples described in more detail below with respect to FIGS. 2-3, the user interface may receive inputs from a user indicating, for example, the location of a feature of interest within an image displayed via the graphical user interface. Further, the graphical user interface may include instructions or other information to guide the user through a process to align the image with respect to the feature of interest. The user interface 115 may include one or more of the following: a rotary, a mouse, a keyboard, a trackball, a touch-sensitive display, hard keys linked to specific actions, soft keys that may be configured to control different functions, and a graphical user interface.

The ultrasound imaging system 100 also includes a processor 116 to control the transmit beamformer 101, the transmitter 102, the receiver 108, and the receive beamformer 110. The processor 116 is in electronic communication with the probe 106. For purposes of this disclosure, the term “electronic communication” may be defined to include both wired and wireless communications. The processor 116 may control the probe 106 to acquire data. The processor 116 controls which of the elements 104 are active and the shape of a beam emitted from the probe 106. The processor 116 is also in electronic communication with a display device 118, and the processor 116 may process the data into images for display on the display device 118. The processor 116 may include a central processor (CPU) according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment the demodulation can be carried out earlier in the processing chain.

The processor 116 is adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire images at a real-time rate of 7-20 volumes/sec. The ultrasound imaging system 100 may acquire 2D data of one or more planes at a significantly faster rate. However, it should be understood that the real-time volume-rate may be dependent on the length of time that it takes to acquire each volume of data for display. Accordingly, when acquiring a relatively large volume of data, the real-time volume-rate may be slower. Thus, some embodiments may have real-time volume-rates that are considerably faster than 20 volumes/sec while other embodiments may have real-time volume-rates slower than 7 volumes/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, 10 Hz to 30 Hz. Images generated from the data may be refreshed at a similar frame-rate. Other embodiments may acquire and display data at different rates. For example, some embodiments may acquire data at a volume-rate of less than 10 Hz or greater than 30 Hz depending on the size of the volume and the intended application. A memory 120 is included for storing processed volumes of acquired data. In an exemplary embodiment, the memory 120 is of sufficient capacity to store at least several seconds worth of volumes of ultrasound data. The volumes of data are stored in a manner to facilitate retrieval thereof according to its order or time of acquisition. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. The image beams and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may be provided that reads the image volumes from a memory and displays an image in real time while a procedure is being carried out on a patient. A video processor module may store the images in the memory 120, from which the images are read and displayed.

As described above, the ultrasound imaging system of FIG. 1 may acquire a plurality of images and construct a three-dimensional volume representing an imaged target region. For example, a fast 3D-sweep acquisition or a spatio-temporal image correlation (STIC) acquisition process may be used to acquire a 3D or 4D volume of a target region, such as a fetal heart. The 3D or 4D volume of the heart may be used to determine a pump volume of the heart, diagnose structural heart defects, or access other desired diagnostic features of the heart. These diagnostic processes may be performed at the time of imaging. In other examples, the volume may be stored and the diagnostic processes may be performed at a time after the imaging. In particular, the image acquisition may be performed by a skilled ultrasound technician, while the diagnostic procedures may be performed by a physician that, while skilled in anatomy and diagnostics, may not be as skilled as the ultrasound technician in ultrasound imaging techniques.

In order to access desired slices of the volume for diagnostic or other purposes, or in order to perform the calculation of the pump volume, the volume of the heart may first be aligned to a standard orientation. Alignment of the volume may be carried out by the technician, or by the physician or less skilled clinician. To ensure the standard alignment may be reached in an easy, fast, and reproducible manner, even if performed by a physician or other clinician not skilled in ultrasound techniques, the ultrasound system of FIG. 1 may include a semi-automatic, guided alignment process that includes aligning three planes of the volume with respect to a feature of interest within the volume, such as the interventricular septum of the heart. The alignment process may include instructions to guide the operator through the alignment as well as automatic rotation and/or translation of the volume based on operator input indicating the location of the feature of interest in each plane.

FIG. 2 is a flow chart illustrating a method 200 for aligning a volume to a standard alignment according to an embodiment of the disclosure. Method 200 may be carried out according to instructions stored on a computing system, including but not limited to the processor and memory of the ultrasound system of FIG. 1. Method 200 includes, at 202, acquiring a plurality of images with an ultrasound probe. The images may be acquired by a suitable probe in a suitable manner, such as a 2D or 3D probe, linear or array transducer probe, in a B-mode, according to a 3D-sweep acquisition or STIC acquisition mode, etc. At 204, method 200 includes constructing a three-dimensional volume based on the plurality of images.

At 206, the three-dimensional model is aligned with respect to a feature of interest within the volume. This may include, as indicated at 208, aligning each plane of three intersecting image planes with respect to the feature of interest. Additional details regarding the aligning of the volume with respect to the feature of interest will be presented below with respect to FIG. 3. Briefly, the alignment includes identifying the feature of interest in at least a first image plane, and adjusting the volume to align the feature of interest to a given axis within the first plane. The volume is adjusted again to align the feature of interest to the given axis in a second plane orthogonal to the first plane. Finally, the volume is adjusted to align the feature of interest to an axis in a third plane, orthogonal to the first and second planes. As the feature of interest may not be visible in the third plane, the alignment in the third plane may instead rely on a different feature visible in the third plane, where the different feature is aligned to a different axis.

At 210, the aligned volume is stored, for example in the memory of the ultrasound system, for future processing. The future processing may include, as indicated at 212, retrieving diagnostically-relevant images, and/or measuring a volume and/or other diagnostically relevant features, as indicated at 214.

Thus, the method described above acquires a plurality of images and constructs a three-dimensional volume from the plurality of images. The volume is then aligned to a standard alignment with respect to a feature of interest of the volume, in three intersecting planes of the volume. Each process described in the method above may be performed with a single ultrasound system in a single imaging session. However, in other examples, the image acquisition and volume construction may be performed by a first ultrasound system and the volume alignment and/or volume processing may be performed by a second, different ultrasound system. Further, in some examples the volume alignment may be performed by a suitable computing system not associated with the ultrasound image acquisition features of the ultrasound system (e.g., the transmitter/receiver).

Accordingly, a method of aligning a volume to a standard orientation includes accessing three images, each image representing a respective plane of three intersecting planes of the volume, identifying a feature of interest in each of the three images, outputting one or more guidance indicators that indicate how the three images are to be aligned with respect to the feature of interest to correspond with the standard orientation, and adjusting the three images according to the one or more guidance indicators.

The one or more guidance indicators may be text and/or graphics that instruct an operator how to adjust the images so that the feature of interest is aligned with a standard alignment. In an example, the volume may represent an anatomical structure, and the feature of interest may be a sub-anatomical structure of the anatomical structure, contained within the anatomical structure. For example, the three accessed images may be images of a heart, such as a fetal heart, and the feature of interest may be an interventricular septum of the heart. In such circumstances, the three images may be adjusted to align the interventricular septum to a respective axis in each of the three intersecting planes. An example method for aligning a three-dimensional model representing a heart with respect to an interventricular septum is presented below with respect to FIG. 3. However, the method 200 described above is not limited to alignment of a heart volume, as other volumes are possible. For example, a volume of a brain may be aligned to a suitable sub-anatomical structure within the brain having a defined and easy to identify orientation, such as the brain stem.

Turning now to FIG. 3, a method 300 for aligning a volume of a heart is presented. Method 300 may be performed by a computing system, such as a computing system of an ultrasound system (e.g., according to instructions stored on the memory of and executed by the processor of the ultrasound system of FIG. 1). Method 300 may be performed in order to align a three-dimensional volume of a heart constructed from a plurality of images acquired by the ultrasound system, as described above with respect to FIG. 2. The alignment process described below includes outputting instructions to a user, via a graphical user interface for example, as well as receiving user inputs made to the graphical user interface. The images of the three planes of the volume manipulated during the alignment are included on the graphical user interface. Accordingly, method 300 will be described along with FIGS. 4-8, which illustrate example graphical user interface outputs during the alignment process.

At 302, method 300 includes outputting instructions indicating that an operator is to identify an interventricular septum in a first plane of the volume. In standard three-dimensional ultrasound systems, when an operator is viewing a three-dimensional volume, typically three image planes of the volume are displayed via a graphical user interface of the ultrasound system: the plane acquired during imaging (e.g., the B plane, also referred to as the transverse plane) and two planes orthogonal to the imaged plane, the A plane (also referred to as the sagittal plane) and the C plane (also referred to as the coronal plane). The displayed planes are relative to the ultrasound probe, and not the patient being imaged, although in some circumstances the planes relative to the probe may be the same as the planes relative to the patient. The instructions may be output via a graphical user interface that may be displayed on a display device of the ultrasound system, for example, or via another suitable method, such as audio outputs.

In order to identify the interventricular septum, the operator first translates and/or rotates the volume until the left and right ventricles are at least partially visible in the A plane. The interventricular septum is defined by the tissue that separates the ventricles, from the apex to the crux of the heart. Thus, the instructions output to indicate to the operator to identify the interventricular septum may include instructions to adjust the volume until the left and right ventricle are at least partially visible in the A plane and also instructions requesting the operator input the exact location within the A plane of the interventricular septum. Further, in some examples, the volume of the heart may be aligned with respect to either the right or left ventricle, and as such the instructions may include instructions for the operator to indicate which ventricle is of interest. While method 300 is described with respect to initially identifying the interventricular septum in the A plane, it is possible that the interventricular septum may be initially located in another plane, such as the C plane.

In some examples, the instructions may include a guidance indicator, e.g., a visual indicator demonstrating to the operator how and/or where to locate the interventricular septum (also referred to herein as the septum). This may include a pictogram schematically showing where the septum is located with respect to the right and left ventricles, or it may include a visual indicator located at the actual location of the septum. The interventricular septum is a sub-anatomical feature of the heart that is easy to identify, even in a developing fetal heart, and that has a defined orientation. Thus, by using the interventricular septum as an orientation landmark, a simple, fast, and reproducible alignment may be achieved, even if the alignment is carried out by a practitioner that is not highly skilled in ultrasound imaging techniques (such as a physician). However, the method is not limited to aligning a heart with respect to an interventricular septum, as other sub-anatomical features of the heart may be used, such as an atrioventricular valve.

FIG. 4 shows an example graphical user interface 400 that may be output on a display device, showing a heart in three planes (the A, B, and C planes) during a first step of the alignment process. Specifically, image 402 is the heart in the A plane, image 404 is the heart in the B plane, and image 406 is the heart in the C plane. The left and right ventricles (LV and RV, respectively) are visible in image 402 (e.g., in the A plane). Additionally, a user selection of a ventricle of interest (the left ventricle) is indicated by a visual indicator, such as dot 408, which is visible in all three planes.

Returning to FIG. 3, at 304, method 300 includes receiving input from the operator indicating the location of the septum as well as a ventricle of interest, in the first plane. The input may comprise a line drawn on the graphical user interface, via a mouse, for example, along a longitudinal axis of the septum from the apex to the crux, as well as a mouse click within the ventricle of interest. FIG. 5 illustrates an example graphical user interface 500 that may be output on a display device during a second step of the alignment. The graphical user interface 500 includes the same images 402, 404, and 406 shown in the graphical user interface 400 of FIG. 4, and additionally shows the user input identifying the longitudinal axis of the septum, in particular as line 502. Further, a visual indicator 504 is included, illustrating where the operator is to place the line to identify the septum (e.g., between the left and right ventricles). As will be explained below, the visual indicator 504 shows the septum aligned along a vertical axis (the vertical axis is vertical with respect to the graphical user interface, for example), and the volume will be rotated to match this alignment.

Thus, at 306, method 300 includes automatically aligning the septum with the vertical axis in the first plane. The volume may be rotated and/or translated until the longitudinal axis of the septum, defined by the line drawn by the operator, is aligned with the vertical axis. This may be performed automatically by the computing system once the operator places the line on the septum, or the volume adjustment to align the septum with the vertical axis may be performed by the operator.

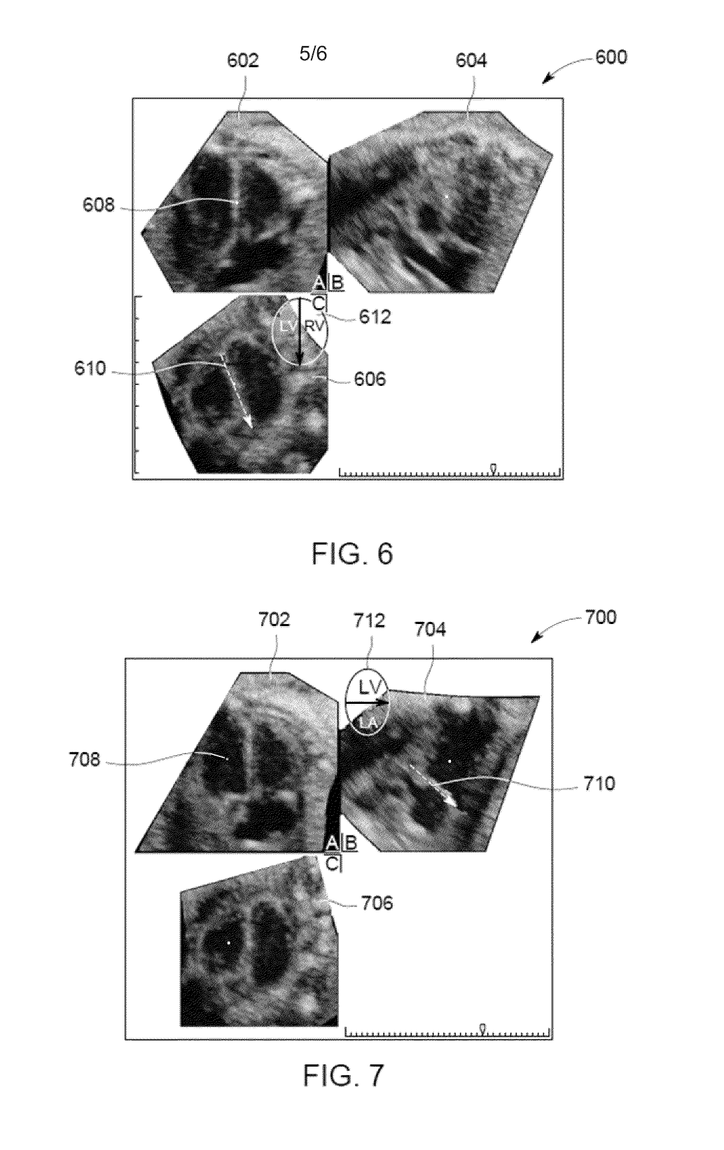

An example graphical user interface illustrating the septum in its rotated position following the alignment of 306 is shown in FIG. 6. Specifically, FIG. 6 shows a graphical user interface 600 that may be output on a display device during a third step of the alignment. The graphical user interface includes three images of the heart, an image 602 in the A plane, an image 604 in the B plane, and an image 606 in the C plane. In image 602, the septum is now aligned along the vertical axis, due to the alignment performed as described above at 306 of method 300. Due to the adjustment (e.g., rotation/translation) of the volume, the images 604 and 606 have also changed with respect to the images 404 and 406, such that the septum is now also visible in image 606.

Returning to FIG. 3, at 308 a visual indicator is output on the graphical user interface indicating the intersection point of the three planes. This visual indicator, which may include a dot in some examples, allows the operator to identify the septum in the second plane of the volume displayed on the graphical user interface (specifically, the C plane). The computing system may automatically place the dot on the graphical user interface, and the dot may be visible in each of the planes. For example, in FIG. 6, the dot 608 represents the location where the three displayed planes intersect, and is typically located in the middle of each image plane.

At 310, method 300 of FIG. 3 outputs instructions indicating that the operator is to identify the septum in the second plane (e.g., the C plane). The instructions for locating the septum in the second plane may be similar to the instructions for locating the septum in the first plane, including a visual indicator illustrating the location of the septum, as well as the axis in the second plane that the septum will be aligned. At 312, the method includes receiving input from the operator indicating a location of the septum in the second plane. Again, as shown in FIG. 6, the input indicating the location may be a line 610 along the longitudinal axis of the septum in image 606. Also shown in FIG. 6 is the visual indicator 612 illustrating the septum and the vertical axis.

At 314, method 300 includes automatically aligning the septum to the vertical axis in the second, C plane, similar to the alignment of the septum to the vertical axis in the first plane described above. As such, the longitudinal axis of the septum will be aligned to the vertical axis. At 316, a visual indicator is output identifying the intersection point of the three planes. The visual indicator is then deviated slightly to one side, either to the right or to the left of the septum, based on the previously input ventricle of interest. For example, if the operator had previously input that the left ventricle is of interest, the visual indicator is placed to the left of the septum (e.g., in the left ventricle). The placement of the visual indicator indicates to the system whether to show the right or the left ventricle in the third plane.

FIG. 7 is an example graphical user interface 700 that may be output on a display device during a fourth step of the alignment process. User interface 700 includes an image 702 in the A plane, an image 704 in the B plane, and an image 706 in the C plane. In the image 702, the heart is aligned similar to the alignment as shown in image 602 of graphical user interface 600, with the septum aligned along the vertical axis of the first plane. In image 706, the septum has been aligned with the vertical axis of the second plane, as described above. Due to the adjustment of the volume in order to align the septum with the vertical axis, as well as the indication of the ventricle of interest, the view of the heart in image 704 has changed relative to the view of image 604. As shown, the left ventricle and the left atrium can be seen in image 704. Further, the visual indicator described above (e.g., the visual indicator deviated horizontally from the 3-plane intersection point) is illustrated as dot 708, which can also be seen in images 704 and 706.

The final portion of the alignment procedure includes aligning a structure within the third plane to an axis. However, the septum, which was used to align the heart in the first and second planes, is not fully visible in the view of the third plane (e.g., the B plane). Thus, instead of aligning the septum, a different structure visible in the third plane is aligned to a different axis. As shown in FIG. 7 and described in more detail below with respect to method 300, an axis (illustrated as line 710) along an atrioventricular (AV) valve between the ventricle and atrium is identified and aligned with respect to a horizontal axis, shown by visual indicator 712. The visible atrioventricular valve may be the mitral valve, as shown in image 704, or the tricuspid valve, if the right ventricle is displayed.

Thus, returning to FIG. 3, at 318, method 300 includes outputting instructions indicating the operator is to identify an AV valve in the third plane (the B plane). At 320, the method includes receiving input form an operator indicting the location of the AV valve in the third plane. At 322, the AV valve is automatically aligned with respect to a horizontal axis in the third plane.

Thus, the method 300 described above uses the interventricular septum of the heart as an anatomical landmark. This landmark is in the heart and thus is not subject to a great amount of change in position relative to other features of the heart among different hearts or patients. A user-friendly, guided, and sequential method prompts the user to place three lines in an easy and fast way and aligns the heart in an exact, reliable, and reproducible manner, tailored to the left or right ventricle to be in focus.

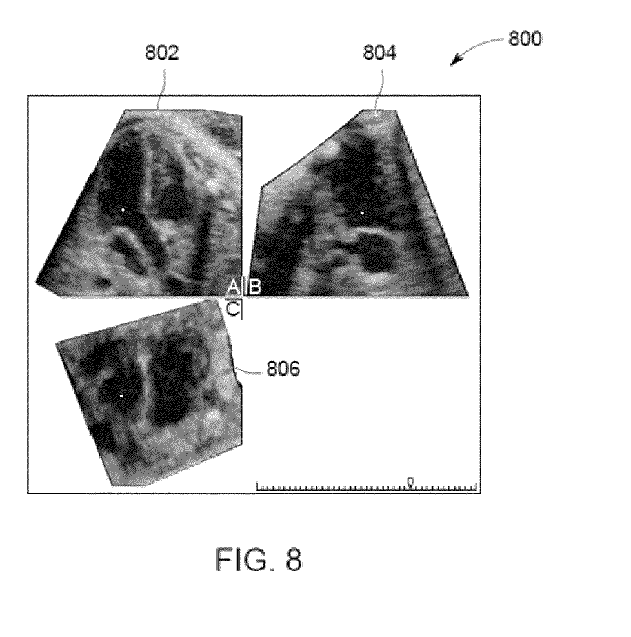

FIG. 8 shows an example graphical user interface 800 that may be output for display on a display device, once the alignment described above is complete. Graphical user interface 800 includes an image 802 in the A plane, showing the septum aligned along a vertical axis in a first, A plane. An image 806 in the C plane shows the septum aligned along a vertical axis in a second, C plane. An image 804 in the B plane shows the AV valve aligned along the horizontal axis in a third, B plane. Due to the adjustment of the volume to bring the AV valve in alignment to the horizontal axis, the views of the heart in the image 802 and image 806 have changed with respect to the images 702 and 706 of graphical user interface 700, although the septum is still visible in each image. Upon completion of the alignment, the volume is in condition for further processing, for example, to calculate the heart pumping volume over time.

The technical effect of the disclosure may include an automatic or semi-automatic alignment of a three-dimensional volume with respect to a feature of interest within the three-dimensional volume. Another technical effect of the disclosure may include the automatic acquisition of diagnostically-relevant images or the calculation of diagnostically relevant features of the aligned three-dimensional volume.

An embodiment relates to a method for automatically aligning a three-dimensional image of a heart. The method includes accessing a three-dimensional volume representing a heart; and aligning the three-dimensional volume with respect to a sub-anatomical structure of the heart. In an example, the method may include acquiring a plurality of images of the heart via a volumetric sweep of an ultrasound transducer and reconstructing the plurality of images into a three-dimensional volume.

The aligning of three-dimensional volume with respect to the sub-anatomical structure of the heart may comprise aligning the sub-anatomical structure to a first axis of a first plane of the three-dimensional volume and aligning the sub-anatomical structure to a second axis of a second plane of the three-dimensional volume. The aligning of the three-dimensional volume with respect to the sub-anatomical structure of the heart may further comprise aligning the three-dimensional volume with respect to an interventricular septum of the heart.

In a first example, aligning the three-dimensional volume with respect to the interventricular septum of the heart may comprise, for a given slice of the three-dimensional volume, aligning a first plane, a second plane, and a third plane of the volume with respect to the interventricular septum. In an example, the given slice comprises a slice including an at least partial view of a left and right ventricle of the heart in the first plane. In a second example, aligning the three-dimensional volume with respect to the interventricular septum of the heart may comprise aligning each plane of a set of orthogonal planes of the volume with respect to the interventricular septum, the set of orthogonal planes comprising a first plane, a second plane, and a third plane. The set of orthogonal planes of the volume may be selected based on a left ventricle and a right ventricle of the heart being at least partially visible in the first plane. In examples, the first plane may be a sagittal plane, the second plane may be a coronal plane, and the third plane may be a transverse plane, with respect to an ultrasound image probe used to acquire the images of the volume.

Aligning the first plane, second plane, and third plane with respect to the interventricular septum comprises first aligning the interventricular septum with a vertical axis in the first plane, then aligning the interventricular septum with a vertical axis in the second plane, then aligning an axis along an atrioventricular valve with a vertical axis in the third plane.

Aligning the interventricular septum with the vertical axis in the first plane may comprise receiving an indication of a location of a longitudinal axis of the interventricular septum in the first plane and rotating the volume until the longitudinal axis of the interventricular septum is aligned with the vertical axis of the first plane. Aligning the interventricular septum with the vertical axis in the second plane may comprise receiving an indication of a location of a longitudinal axis of the interventricular septum in the second plane and rotating the volume until the longitudinal axis of the interventricular septum is aligned with the vertical axis of the second plane.

Aligning the axis along the atrioventricular valve with the horizontal axis in the third plane may comprise receiving an indication of a location of the axis along the atrioventricular valve in the third plane and rotating the volume until the axis along the atrioventricular valve is aligned with the horizontal axis of the third plane. The method may include displaying either a left or a right ventricle in the third plane based on user input.

Another embodiment relates to a system. The system comprises an ultrasound probe to emit ultrasonic signals; an ultrasound receiver to receive echoes of the emitted ultrasonic signals; and a computing system operably connected to the ultrasound probe, ultrasound receiver, and a display device. The computing system includes instructions executable by a processor to: acquire a plurality of images of anatomical structure via a volumetric sweep of the ultrasound probe; reconstruct the plurality of images into a three-dimensional volume; and align the three-dimensional volume with respect to a feature within the anatomical structure.

The computing system may include instructions to output a graphical user interface for display on the display device, the graphical user interface including at least three orthogonal image planes of the three-dimensional volume. The graphical user interface may further comprise visual indicators to guide an operator through one or more steps of the alignment.

In an example, the feature is a sub-anatomical structure of the anatomical structure, and the instructions to align the volume with respect to sub-anatomical structure may include instructions to: output a visual indicator on the graphical user interface instructing the operator to identify the sub-anatomical structure in a first plane of the three image planes, and automatically rotate the volume to align the sub-anatomical structure with a first axis in the first plane.

The instructions may include instructions to: responsive to the sub-anatomical structure being aligned with the first axis in the first plane, output a visual indicator on the graphical user interface instructing the operator to identify the sub-anatomical structure in a second plane of the three image planes, and automatically rotate the volume to align the sub-anatomical structure with a second axis in the second plane.

The instructions may include instructions to: responsive to the sub-anatomical structure being aligned with the second axis in the second plane, output a visual indicator on the graphical user interface instructing the operator to identify a different sub-anatomical structure in a third plane of the three image planes, and automatically rotate the volume to align the different sub-anatomical structure with a third axis in the third plane.

The sub-anatomical structure is contained within (e.g., is a part of) the anatomical structure. As such, the sub-anatomical structure does not comprise the entire anatomical structure. Likewise, the entire anatomical structure does not only comprise the sub-anatomical structure, but also includes additional sub-anatomical structures. In one example, the anatomical structure is comprised of two or more volumes that make up the entire anatomical structure, and the three-dimensional volume may be aligned with respect to a sub-anatomical structure defining one or more of the volumes of the anatomical structure, such as a wall between two volumes.

In an example, the anatomical structure is a heart, the sub-anatomical structure is an interventricular septum, and the different sub-anatomical structure is an atrioventricular valve. The first axis is a vertical axis, the second axis is a vertical axis, and the third axis is a horizontal axis.

A further embodiment relates to a method for aligning a fetal heart volume, comprising: generating a three-dimensional volume representing a heart of a fetus based on a plurality of ultrasound images; semi-automatically aligning the volume to a standard alignment by aligning an interventricular septum of the volume to a vertical axis in one or more planes of the volume, the semi-automatic alignment including outputting notifications to a user to guide the user through the alignment, receiving an indication of a location of the interventricular septum, and automatically rotating the volume in response to receiving the indication; and identifying at least one diagnostically-relevant plane of the aligned volume for output on a display device.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal language of the claims.

1. A method of aligning a volume to a standard orientation, comprising:
accessing three images, each image representing a respective plane of three intersecting planes of the volume;
identifying a feature of interest in each of the three images;
outputting one or more guidance indicators that indicate how the three images are to be aligned with respect to the feature of interest to correspond with the standard orientation; and
adjusting the three images according to the one or more guidance indicators.

2. The method of claim 1, wherein accessing three images comprises accessing three images of a heart.

3. The method of claim 2, wherein the heart is a fetal heart.

4. The method of claim 2, wherein identifying a feature of interest in each of the three images comprises identifying an interventricular septum.

5. The method of claim 4, wherein adjusting the three images comprises adjusting the three images to align the interventricular septum to a respective axis in each of the three intersecting planes.

6. A method for automatically aligning a three-dimensional image of a heart, comprising:
accessing a three-dimensional volume representing a heart; and
aligning the three-dimensional volume with respect to a sub-anatomical structure of the heart.

7. The method of claim 6, wherein aligning the three-dimensional volume with respect to the sub-anatomical structure of the heart comprises aligning the sub-anatomical structure to a first axis of a first plane of the three-dimensional volume and aligning the sub-anatomical structure to a second axis of a second plane of the three-dimensional volume.

8. The method of claim 6, wherein aligning the three-dimensional volume with respect to the sub-anatomical structure of the heart comprises aligning the three-dimensional volume with respect to an interventricular septum of the heart.

9. The method of claim 8, wherein aligning the three-dimensional volume with respect to the interventricular septum of the heart comprises, aligning each plane of a set of orthogonal planes of the volume with respect to the interventricular septum, the set of orthogonal planes comprising a first plane, a second plane, and a third plane.

10. The method of claim 9, wherein aligning each plane of the set of orthogonal planes with respect to the interventricular septum comprises first aligning the interventricular septum with a vertical axis in the first plane, then aligning the interventricular septum with a vertical axis in the second plane, then aligning an axis along an atrioventricular valve with a horizontal axis in the third plane.

11. The method of claim 10, wherein aligning the interventricular septum with the vertical axis in the first plane comprises receiving an indication of a location of a longitudinal axis of the interventricular septum in the first plane and rotating the volume until the longitudinal axis of the interventricular septum is aligned with the vertical axis of the first plane.

12. The method of claim 10, wherein aligning the interventricular septum with the vertical axis in the second plane comprises receiving an indication of a location of a longitudinal axis of the interventricular septum in the second plane and rotating the volume until the longitudinal axis of the interventricular septum is aligned with the vertical axis of the second plane.

13. The method of claim 10, wherein aligning the axis along the atrioventricular valve with the horizontal axis in the third plane comprises receiving an indication of a location of the axis along the atrioventricular valve in the third plane and rotating the volume until the axis along the atrioventricular valve is aligned with the horizontal axis of the third plane.

14. The method of claim 13, further comprising displaying either a left or a right ventricle in the third plane based on user input.

15. The method of claim 9, wherein the set of orthogonal planes of the volume is selected based on a left ventricle and a right ventricle of the heart being at least partially visible in the first plane.

16. A system, comprising:
an ultrasound probe to emit ultrasonic signals;
an ultrasound receiver to receive echoes of the emitted ultrasonic signals; and
a computing system operably connected to the ultrasound probe, ultrasound receiver, and a display device, the computing system including instructions to:
acquire a plurality of images of an anatomical structure via a volumetric sweep of the ultrasound probe;
reconstruct the plurality of images into a three-dimensional volume; and
align the three-dimensional volume with respect to a feature within the anatomical structure.

17. The system of claim 16, wherein the computing system includes instructions to output a graphical user interface for display on the display device, the graphical user interface including at least three orthogonal image planes of the three-dimensional volume.

18. The system of claim 17, wherein the graphical user interface further comprises visual indicators to guide an operator through one or more steps of the alignment.

19. The system of claim 18, wherein the feature is a sub-anatomical structure within the anatomical structure, and wherein the instructions to align the volume with respect to the sub-anatomical structure include instructions to:
output a first visual indicator on the graphical user interface instructing the operator to identify the sub-anatomical structure in a first plane of the three image planes,
automatically rotate the volume to align the sub-anatomical structure with a first axis in the first plane,
responsive to the sub-anatomical structure being aligned with the first axis in the first plane, output a second visual indicator on the graphical user interface instructing the operator to identify the sub-anatomical structure in a second plane of the three image planes,
automatically rotate the volume to align the sub-anatomical structure with a second axis in the second plane,
responsive to the sub-anatomical structure being aligned with the second axis in the second plane, output a third visual indicator on the graphical user interface instructing the operator to identify a different sub-anatomical structure within the anatomical structure in a third plane of the three image planes, and
automatically rotate the volume to align the different sub-anatomical structure with a third axis in the third plane.

20. The system of claim 19, wherein the anatomical structure is a heart, the sub-anatomical structure is an interventricular septum, and the different sub-anatomical structure is an atrioventricular valve, and wherein the first axis is a vertical axis, the second axis is a vertical axis, and the third axis is a horizontal axis.
